(19) United States
(12) Patent Application Publication
(10) Pub. No.: US 2025/0176557 A1
(43) Pub. Date: Jun. 5, 2025

(54) Materials and Methods for Control of Iron-Capturing Pathogens

(71) Applicant: Locus Solutions IPCo, LLC, Solon, OH (US)

(72) Inventor: Maja MILOVANOVIC, North Royalton, OH (US)

(21) Appl. No.: 18/843,550

(22) PCT Filed: Jul. 7, 2023
(86) PCT No.: PCT/US2023/069760

§ 371 Date: Sep. 3, 2024

Related U.S. Application Data
(60) Provisional application No. 63/359,248, filed on Jul. 8, 2022.

(51) Int. Cl. A01N 63/22

(52) U.S. Cl. CPC A01N 63/22

(57) ABSTRACT

The subject invention provides compositions and methods for controlling iron-capturing pathogens. In preferred embodiments, the subject invention can be applied to an environment in which a pathological microorganism is growing, wherein the microorganism pathologically captures iron in order to control the pathogen by outcompeting the pathogen for iron resources.

### CROSS-REFERENCE TO RELATED APPLICATION

This application claims priority to U.S. Provisional Application No. 63/359,248, filed Jul. 8, 2022, which is incorporated herein by reference in its entirety.

### BACKGROUND OF THE INVENTION

Iron-capturing pathogens are bacterial pathogens that can acquire iron through, e.g., receptor-mediated recognition of transferrin, lactoferrin, hemopexin, hemoglobin, or hemoglobin-haptoglobin complexes.

Vertebrate animals do not contain free iron, meaning that all bacterial pathogens experience a period of iron deprivation upon entering a host. Iron is essential for bacterial and fungal physiological processes, such as, for example, DNA replication, transcription, metabolism, and respiration. Thus, pathogens rely on iron assimilation as a virulence factor in many ways. They can use different strategies to obtain iron directly from a host, including, for example, the production of extracellular Fe³⁺ chelating molecules, the uptake of heme and the uptake of Fe²⁺.

Some iron-capturing pathogens include, for example, E. coli, Vibrio cholerae, Salmonella enterica, Streptococcus spp., Yersinia pestis, Erwinia amylovora, Haemophilus influenzae, Dickeya dadantii, Klebsiella pneumoniae, Staphylococcus aureus, Mycobacterium tuberculosis, Legionella pneumophila, Neisseria meningitidis, Bartonella quintana, Bacillus anthracis, Pseudomonas syringae and Serratia marcescens.

As a specific example, Legionella pneumophila, is an intracellular pathogenic bacterium that causes Legionnaires' disease, a serious form of pneumonia. The disease is often contracted when a subject breathes in droplets of water or swallows water containing the bacterium. L. pneumophila cannot survive without iron. Iron is also key in the microbes pathogenesis and can capture iron from its host's cells through multiple specialized metabolic pathways.

An additional example, Neisseria spp. (e.g., N. meningitidis and N. gonorrhoeae), can cause diseases such as meningitis, septicemia and gonorrhea. These bacteria have developed mechanisms to capture iron from host proteins, such as lactoferrin and transferrin.

Plants can also be affected by iron-capturing pathogens. Dickeya dadantii, for example, is a soft-rotting enterobacterium that attacks a wide range of plant species, including many vegetables and ornamentals, through degradation of pectin. These bacteria are found in soil and on plant surfaces, and often enter a plant via wound sites or through natural openings. Production of siderophores by D. dadantii allows the microbe to acquire iron from the host plant and to promote systemic infection.

Thus, bacterial infections are a widespread issue in human and veterinary medicine, as well as agriculture. Antibiotics serve as the most common tool for combatting infections; however, their overuse has led to growing concern over the development of antibiotic-resistant strains of microorganisms. Even further, many antibiotics have low or non-existence efficacy against infections that are present in a biofilm state.

Bacteria in a biofilm respond collectively to certain factors that are specific to the biofilm phenotype, which lead to the secretion of an exopolysaccharide (EPS) matrix surrounding and connecting the individual cells. Biofilms behave differently from the same bacteria in free-floating form. They are far less susceptible to antibiotics, making certain infections, such as pneumonia, difficult to treat—and potentially lethal. Furthermore, because antibiotics fail to eradicate these EPS-protected microbial communities, use of antibiotics can compound the problem because antibiotics select for, and perpetuate, increasingly antibiotic-resistant bacteria. These bacteria include methicillin-resistant Staphylococcus aureus (MRSA), the world's leading cause of nosocomial infection, and a bacterium now widespread in the community at large.

Iron-capturing bacteria can be the cause of a range of difficult-to-treat diseases and health conditions in humans, animals and plants. Iron, or lack thereof, is a fundamental sensory cue in bacterial pathogens, and it can trigger the coordinated regulation of genes involved in both iron acquisition and virulence. Furthermore, biofilms can exacerbate the virulence and difficulties in treating these pathogens. Therefore, new compositions and methods are needed for treating infections in the body, in plants, and on equipment in hospital, clinics, and operating rooms.

### BRIEF SUMMARY OF THE INVENTION

The subject invention provides compositions and methods for controlling iron-capturing pathogens. More specifically, the subject invention provides compositions that, when applied to an environment in which an iron-capturing pathological microorganism is growing, can regulate the pathogen in a way that reduces and/or eliminates its pathogenicity. Advantageously, the compositions and methods can be useful for treating and/or preventing diseases and infections caused by iron-capturing pathogens.

In preferred embodiments, the subject invention provides methods for controlling iron-capturing pathogens, wherein a composition comprising an iron-capturing ingredient is introduced into an environment in which the pathological microorganism is growing. Thus, the subject invention provides methods for controlling a pathogen, wherein the composition applied to the pathogen's environment outcompetes the pathogen for iron resources. The methods can be utilized in environments including, for example, human and/or animal subjects, soil, plants, water and/or inanimate surfaces.

Advantageously, in certain embodiments, the methods result in capture of iron present in the pathogen's environment, thereby inhibiting its pathogenicity via iron limitation. Thus, in preferred embodiments, the methods result in a direct inhibition of pathogens, increased out-competition of pathogens by beneficial microorganisms, disruption of pathogenic biofilms, and/or disruption of one or more biological pathways in which the pathogen utilizes iron for growth, reproduction and/or virulence.

In one embodiment, the method of the subject invention comprises identifying the presence of an iron-capturing pathogen prior to administering the composition of the subject invention.

Non-limiting examples of bacterial pathogens that can utilize one or more iron acquisition systems to enhance infectious virulence include Escherichia spp. (e.g., E. coli), Vibrio spp. (e.g., V. cholerae, V. vulnificus), Shigella spp. (e.g., S. flexneri, S. dysenteriae), Salmonella spp. (e.g., S. enterica), Streptococcus spp. (e.g., S. puyogenes, S. agalactiaei, Group A Streptococcus), Yersinia . . . spp. (e.g., Y. pestis), Haemophilus spp. (e.g., H. influenzae), Klebsiella spp. (e.g. K. pneumoniae), Staphylococcus spp. (e.g., S. aureus), Mycobacterium spp. (e.g., M. tuberculosis), Neisseria spp. (e.g., N. meningitidis, N. gonorrhoeae), Bartonella spp. (e.g., B. quintana), Bacillus spp. (e.g., B. anthracis, B. cereus), Serratia spp. (e.g., S. marcescens), Pseudomonas spp. (e.g., P. aeruginosa), Legionella spp. (e.g., L. pneumophila), Meningococcus spp., Brucella spp. (e.g., B. abortus), Listeria spp. (e.g., L. monocytogenes), Acinetobacter spp. (e.g., A. baumannii), Francisella spp. (e.g., F. tularensis), and/or Haemophilus spp. (e.g., H. influenzae).

The subject invention further provides a composition comprising one or more ingredients that capture iron. In certain embodiments, the iron-capturing ingredient is a beneficial microorganism, a growth by-product of an iron-capturing microorganism, or some other compound known to bind iron. In certain embodiments, more than one iron-capturing ingredient is included in the composition.

In preferred embodiments, the beneficial microorganisms of the subject invention are non-pathogenic fungi, yeasts and/or bacteria capable of sequestering iron, either naturally or through genetic modification.

In one embodiment, the beneficial microorganism is a strain of Bacillus subtilis. In a specific embodiment, the strain is B. subtilis B4 (NRRL B-68031). The B4 strain is preferably administered in spore form but grows in biofilm form when exposed to acidic environments.

Surprisingly, B4 was found to produce one or more compounds capable of sequestering, chelating or otherwise capturing iron. In certain embodiments, the compounds are pulcherrimin and/or pulcherriminic acid. Advantageously, the microbes and/or the exopolysaccharide (EPS) of the microbes when grown in biofilm form effectively hoard freely-available iron using an iron-capturer such as pulcherrimin and/or pulcherriminic acid upon exposure to an acidic pH (e.g., less than 6.8, preferably less than 5.0).

B4 is also particularly advantageous over other traditional probiotic microorganisms due to its ability to produce digestive enzymes, including, for example, cellulases and amylases.

In certain embodiments, the composition can comprise other non-pathogenic microorganisms that are capable of producing compounds that can sequester, chelate or otherwise capture iron. The microorganism(s) can be in biofilm form, spore form, planktonic form, or any other form.

In certain embodiments, the microorganisms are also capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as amylases, cellulases, proteases and/or lipases; and sources of amino acids, vitamins, and other nutrients.

In certain embodiments, the iron-capturing ingredient of the subject composition is a crude form or purified siderophore or phytosiderophore, or other molecule with high iron affinity, for example, pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine.

In certain embodiments, the composition comprises an organic or inorganic acid. Preferably, the acid is present in an amount suitable for adjusting the pH of the composition or the environment to which it is applied to 6.8 or lower, preferably, 5.0 or lower, more preferably, 4.8 or lower.

In certain embodiments, the composition can be formulated for internal and/or external administration to a human or animal subject, e.g., as an orally-consumable formulation, a topical formulation, or an injectable formulation. In other embodiments, the composition can be formulated for application to a plant or to soil, e.g., as a powder or liquid. In yet other embodiments, the composition can be formulated for application to water and/or to inanimate or inert surfaces, e.g., as a liquid, gel, powder or aerosol.

### BRIEF DESCRIPTION OF THE FIGURES

FIGS. 1A-1C show growth and exopolysaccharide (EPS) formation of B4 after 24 hours (A) and 48 hours (B) at acidic pH (top row, pH 4.8) and neutral pH (bottom row, pH 6.8).

FIG. 2 shows purified B4 EPS with a pink hue.

FIG. 3 shows result of an amylase test for B4. Agar streaked with B4 produced an orange color around the bacterial growth, indicating the breakdown of starch.

FIG. 4 show results of a cellulase test for B4. Agar streaked with B4 produced a yellow zone of clearing around the bacterial growth, indicating the breakdown of cellulose.

FIGS. 5A-5C show B4 siderophore production and activity after 6 hours in aerobic (top plates) and anaerobic (bottom plates) environments and on different growth media. (A) shows B4 culture grown in MRS-sucrose (left side) and M23-6 (right side) media. (B) shows B4 culture grown in minimal media with Tween (left side) and minimal media without Tween (right side). (C) shows dried B4 spores grown in minimal medium.

FIGS. 6A-6C show B4 siderophore production and activity after 24 hours in aerobic (top plates) and anaerobic (bottom plates) environments and on different growth media. (A) shows B4 culture grown in M23-6 (left side) and MRS-sucrose (right side) media. (B) shows B4 culture grown in minimal media with Tween (left side) and minimal media without Tween (right side). (C) shows dried B4 spores grown in minimal medium.

FIG. 7 shows results of an iron assay for B4 (BSSL) cultures grown in different media.

### DETAILED DESCRIPTION OF THE INVENTION

The subject invention provides compositions and methods for controlling iron-capturing pathogens. More specifically, the subject invention provides compositions that, when applied to an environment in which a pathological microorganism is growing, can regulate the iron-capturing pathogen in a way that reduces and/or eliminates its pathogenicity.

Advantageously, in some embodiments, the compositions and methods can also be used to treat and/or prevent a disease or infection of a human, plant or animal that is caused by an iron-capturing pathogen.

### Selected Definitions

As used herein, a “biofilm” is a complex aggregate of microorganisms, such as bacteria, wherein the cells adhere to each other and/or to a surface. In certain embodiments, adherence is achieved via an exopolysaccharide substance produced by the bacteria. The cells in biofilms are physiologically distinct from planktonic cells of the same organism, which are single cells that can float or swim in liquid medium.

As used herein, the term “control” used in reference to an undesirable microorganism (e.g., a pathogen) extends to the act of killing, disabling, immobilizing and/or reducing the population numbers of the microorganism, and/or otherwise rendering the microorganism incapable of reproducing and/or carrying out the processes that are undesirable (e.g., infectious pathogenicity).

As used herein, “infection” refers to the introduction and/or presence of a disease-causing, or pathogenic, organism into and/or in another organism, tissue or cell.

As used herein “preventing” or “prevention” of a disease, condition or disorder means delaying, inhibiting, suppressing, forestalling, and/or minimizing the onset or progression of a particular sign or symptom thereof. Prevention can include, but does not require, indefinite, absolute or complete prevention throughout a subject's lifetime, meaning the sign or symptom may still develop at a later time. Prevention can include reducing the severity of the onset of such a disease, condition or disorder, and/or inhibiting the progression of the condition or disorder to a more severe condition or disorder.

As used herein, “treating” or “treatment” of a disease, condition or disorder means the eradicating, improving, reducing, ameliorating or reversing of at least one sign or symptom of the disease, condition or disorder (e.g., an infection). Treatment can include, but does not require, a complete cure of the disease, condition or disorder, meaning treatment can also include partial eradication, improvement, reduction, amelioration or reversal.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “ionophores” are carboxylic polyether non-therapeutic antibiotics that disrupt the ion concentration gradient (Ca2+, K+, H+, Na+) across microorganisms, which causes them to enter a futile ion cycle. The disruption of the ion concentration prevents the microorganism from maintaining normal metabolism and causes the microorganism to expend extra energy. Ionophores function by selecting against or affecting the metabolism of gram-positive bacteria, such as methanogens, and protozoa.

As used herein, “siderophores” are compounds produced by different organisms for the purpose of scavenging iron from the surrounding environment. Siderophores are typically small, low molecular weight compounds with high affinity for ferric iron (Fe³⁺), forming strong ferric chelate complexes that can, in some instances be taken up by the organisms. As used herein, “phytosiderophores” are siderophores produced by plants.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, polyketides, and surfactants.

As used herein, a “methanogen” is a microorganism that produces methane gas as a by-product of metabolism. Methanogens are archaea that can be found in the digestive systems and metabolic waste of ruminant animals and non-ruminant animals (e.g., pigs, poultry and horses). Examples of methanogens include, but are not limited to, Methanobacterium spp. (e.g., M. formicicum), Methanobrevibacter spp. (e.g., M. ruminantium), Methanococcus spp. (e.g., M. paripaludis), Methanoculleus spp. (e.g., M. bourgensis), Methanoforens spp. (e.g., M. stordalenmirensis), Methanofollis liminatans, Methanogenium wolfei, Methanomicrobium spp. (e.g., M. mobile), Methanopyrus kandleri, Methanoregula boonei, Methanosaeta spp. (e.g., M. concilii, M. thermophile), Methanosarcina spp. (e.g., M. barkeri, M. mazeii), Methanosphaera stadtmanae, Methanospirillium hungatei, Methanothermobacter spp., and/or Methanothrix sochngenii.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” means a negative alteration and “increase” means a positive alteration, wherein the positive or negative alteration is at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Compositions

In preferred embodiments, the subject invention provides methods for controlling iron-capturing pathogens, wherein a composition comprising an iron-capturing ingredient is introduced into an environment in which the pathological microorganism is growing. Thus, the subject invention provides methods for controlling a pathogen, wherein the composition applied to the pathogen's environment outcompetes the pathogen for iron resources. The methods can be utilized in environments including, for example, human and/or animal subjects, soil, plants, water and/or inanimate surfaces.

The subject invention further provides a composition for use according to the subject methods, wherein the composition comprises one or more ingredients that capture iron. In certain embodiments, the iron-capturing ingredient is a beneficial microorganism, a growth by-product of an iron-capturing microorganism, or some other compound known to bind iron. In certain embodiments, more than one iron-capturing ingredient is included in the composition.

The total iron-capturing ingredient(s) in the composition preferably comprise from 0.0001% to 100% of the composition by weight or by volume, or from 0.001 to 95%, from 0.01 to 90%, from 0.1% to 85%, from 0.5 to 80%, from 0.75 to 75%, from 1.0 to 70%, from 1.25 to 65%, from 1.5 to 60%, from 1.75 to 55%, from 2.0 to 50%, or from 5.0 to 25% by weight or by volume.

In certain embodiments, the composition is a “microbe-based composition,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The cells may be totally absent, or present at, for example, a concentration of at least 1×10⁴, 1×10⁵, 1×10⁶, 1×10⁷, 1×10⁸, 1×10⁹, 1×10¹⁰, 1×10¹¹, 1×10¹², 1×10¹³ or more CFU per milliliter or CFU/g of the composition.

Advantageously, in preferred embodiments, the subject compositions can alter metabolic pathways of iron-capturing pathogens, resulting in decreased activity, virulence and/or pathogenicity.

In some embodiments, the composition can also be used for cleaning inert surfaces containing an iron-capturing pathogen. Advantageously, in some embodiments, this can result in reduced occurrences of infection caused by the pathogen.

In preferred embodiments, the beneficial microorganisms of the subject compositions are non-pathogenic fungi, yeasts and/or bacteria capable of sequestering iron, either naturally or through genetic modification. The beneficial microorganisms may be in an active, inactive and/or dormant form. In preferred embodiments, the microorganism is one that is characterized as “generally regarded as safe,” or GRAS, by the appropriate regulatory agency.

In certain embodiments, the microorganisms are also capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as amylases, cellulases, proteases and/or lipases; and sources of amino acids, vitamins, and other nutrients.

The microorganisms of the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant, or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In some embodiments, the beneficial microorganisms are selected based on a natural or acquired resistance to certain antibiotics administered to an environment comprising an iron-capturing pathogen to, for example, control pathogenic and/or deleterious microbes in a living subject or elsewhere in an environment.

In one specific embodiment, the composition comprises about 1×10⁶ to about 1×10¹³, about 1×10⁷ to about 1×10¹², about 1×10⁸ to about 1×10¹¹, or about 1×10⁹ to about 1×10¹⁰ CFU/g of each species of microorganism present in the composition.

In one embodiment, the composition comprises about 0.001 to 100% microorganisms total by volume, about 1 to 90%, or about 10 to 75%.

In certain embodiments, the composition comprises a growth by-product of a microorganism but no living microorganism. For example, in certain embodiments, a pathogenic microorganism is utilized only in the production of growth by-products for producing a composition according to the subject invention as opposed to direct administration to an environment.

The microorganisms can include yeasts, bacteria and/or fungi, including, for example, Acaulospora, Acidithiobacillus spp. (e.g., A. ferooxidans, A. albertensis, A. caldus, A. cuprithermicus, A. ferrianus, A. ferridurans, A. ferriphilus, A. ferrivorans, A. ferrooxidans, A. sulfuriphilus, and A. thiooxidans), Acremonium chrysogenum, Agrobacterium (e.g., A. radiobacter), Aspergillus, Aureobasidium (e.g., A. pullulans), Azospirillum (e.g., A. brasiliensis), Azotobacter (A. vinelandii, A. chroococcum), Bacillus (e.g., B. amyloliquefaciens, B. coagulans, B. firmus, B. laterosporus, B. licheniformis, B. megaterium, B. mucilaginosus, B. subtilis), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Dipodascopsism, Entomophthora, Escherichia coli, Frateuria (e.g., F. aurantia), Hanseniaspora (e.g., H. uvarum), Hansenula, Issatchenkia, Kluyveromyces (e.g., K. phaffii), Lentinula spp. (e.g., L. edodes), Legionella pneumophila, Lipomyces, Magnetospirillum magneticum, Magnetococcus marinus, methanogens, Metschnikowia sp. (M. pulcherrimia), Meyerozyma (e.g., M. guilliermondii, M. caribbica), Monascus purpureus, Mortierella, Mucor (e.g., M. piriformis), Neisseria meningitidis, Pantoea (e.g., P. agglomerans, P. allii), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudomonas (e.g., P. chlororaphis, P. aeruginosa, P. koreensis), Pseudozyma (e.g., P. aphidis, P. antarctica), Rhizobium radiobacter, Rhizopus, Rhodospirillum (e.g., R. rubrum), Rhodotorula (e.g., R. bogoriensis), Saccharomyces (e.g., S. cerevisiae, S. boulardii, S. torula), Sphingomonas (e.g., S. paucimobilis), Starmerella (e.g., S. bombicola), Streptomyces, Torulopsis, Thraustochytrium, Trichoderma (e.g., T. reesei, T. harzianum, T. viridae), Ustilago (e.g., U. maydis), Vibrio cholerae, Wickerhamiella (e.g., W. domericqiae), Wickerhamomyces (e.g., W. anomalus), Williopsis (e.g., W. mrakii), Zygosaccharomyces (e.g., Z. bailii), and others (including those listed as pathogens elsewhere in this disclosure).

If present, fungi can be in the form of live or inactive cells, mycelia, spores and/or fruiting bodies. The fruiting bodies, if present, can be, for example, chopped and/or blended into granules and/or a powder form.

If present, yeasts can be in the form of live or inactive cells or spores, as well as in the form of dried and/or dormant cells (e.g., a yeast hydrolysate).

If present, bacteria can be in the form of vegetative or planktonic cells, biofilms, spores, and/or a dried cell or spore mass.

In some embodiments, dried microbes, e.g., spores, can be mixed with fillers known in the art, such as e.g., microcrystalline cellulose (MCC).

In one embodiment, the composition comprises one or more Bacillus spp. bacteria and/or growth by-products thereof. In certain embodiments, the Bacillus spp. are B. amyloliquefaciens, B. subtilis, B. coagulans and/or B. licheniformis.

In one embodiment, the composition comprises B. amyloliquefaciens NRRL B-67928 “B. amy” and/or a growth by-product thereof. A culture of the B. amyloliquefaciens “B. amy” microbe has been deposited with the Agricultural Research Service Northern Regional Research Laboratory (NRRL) Culture Collection, 1815 N. University St., Peoria, IL, USA. The deposit has been assigned accession number NRRL B-67928 by the depository and was deposited on Feb. 26, 2020.

In one embodiment, the composition comprises a strain of Bacillus subtilis and/or a growth by-product thereof. In a specific embodiment, the strain is B. subtilis B4 (NRRL B-68031). A culture of the B4 microbe has been deposited with the Agricultural Research Service Northern Regional Research Laboratory (NRRL) Culture Collection, 1815 N. University St., Peoria, IL, USA. The deposit has been assigned accession number NRRL B-68031 by the depository and was deposited on May 6, 2021.

B4 is a Gram-positive spore-forming strain of B. subtilis that is capable of anaerobic growth (obligate anaerobe). The B4 strain is preferably administered in spore form but germinates in acidic environments, wherein it can grow in biofilm form. Surprisingly, B4 was found to produce one or more compounds capable of sequestering, chelating or otherwise capturing iron when grown in biofilm form. In certain embodiments, the compounds are pulcherrimin and/or pulcherriminic acid.

Advantageously, the microbes and/or the exopolysaccharide (EPS) of the biofilm effectively hoard freely-available iron using an iron-capturer such as, e.g., pulcherrimin and/or pulcherriminic acid, while traveling through low pH (e.g., less than 6.8, less than 5.0, or less than 4.8).

B4 is also particularly advantageous over other traditional probiotic microorganisms when administered to a living subject due to its ability to produce increased amounts of the lipopeptide surfactin (e.g., greater than wild type B. subtilis), as well as digestive enzymes, including, for example, cellulases and amylases. These enzymes help digest nutrient matter into smaller units, such as volatile fatty acids (e.g., propionate, acetate, butyrate), glucose and amino acids.

The proprietary cultures described herein have been deposited under conditions that assure that access to the cultures will be available during the pendency of this patent application to one determined by the Commissioner of Patents and Trademarks to be entitled thereto under 37 CFR § 1.14 and 35 U.S.C § 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, each of the subject culture deposits will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In certain embodiments, the composition can comprise other microorganisms that are capable, either naturally or by genetic modification, of producing pulcherrimin and/or pulcherriminic acid, or other compounds capable of sequestering, chelating or otherwise capturing iron. In specific embodiments, the microbes are capable of growing as a biofilm.

In certain embodiments, the microorganism is a naturally-occurring or genetically-modified microorganism capable of regulating genes involved in iron capture and transport, e.g., HFE, GDF15, TWSG1, ERFE, Matriptase 2, TF, TFR1, TFR2, HAMP and HJV.

In certain embodiments, the composition can comprise a crude form or purified siderophore or phytosiderophore, or other molecule with high iron affinity, for example, pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine.

The composition can also comprise other microbial growth by-products. The microbial growth by-product can be produced by the microorganisms of the composition, and/or they can be produced separately, e.g., by a microorganism listed herein, and added to the composition.

In certain embodiments, the composition can comprise substrate leftover from cultivation, and/or purified or unpurified growth by-products, such as biosurfactants, killer toxins, enzymes, polyketides, and/or other metabolites. The microbes can be live or inactive, although, in preferred embodiments, if the microbe is considered a pathogen, the microbe is inactivated and/or removed from the composition.

In one embodiment, the growth by-product has been purified from the cultivation medium in which it was produced. Alternatively, in one embodiment, the growth by-product is utilized in crude form. The crude form can comprise, for example, a liquid supernatant resulting from cultivation of a microbe that produces the growth by-product of interest, including residual cells and/or nutrients.

The growth by-products can include metabolites or other biochemicals produced as a result of cell growth, including, for example, amino acids, peptides, polyketides, antibiotics, proteins, enzymes, biosurfactants, solvents, vitamins, and/or other metabolites.

The microorganism(s) and/or growth by-product(s) present in the composition can be useful for inhibiting pathogens and/or biological pathways that contribute to their pathogenicity and/or virulence, disrupting pathogen biofilms, and/or reducing H₂ accumulation in an iron-capturing pathogen's environment.

### Additional Components

In certain embodiments, the composition comprises a germination enhancer for enhancing germination of spore-form microorganisms used in the microbe-based composition. In specific embodiments, the germination enhancers are amino acids, such as, for example, L-alanine and/or L-leucine. In one embodiment, the germination enhancer is manganese.

In certain embodiments, the composition comprises an organic acid selected from, for example, acetic acid, citric acid, formic acid, lactic acid, malic acid, oxalic acid, tartaric acid, uric acid, propionic acid, butyric acid, sorbic acid, fumaric acid, benzoic acid, hydrofluoric acid, caproic acid, salicylic acid, gluconic acid, pyruvic acid, adipic acid, trichloroacetic acid, glycolic acid, cinnamic acid, carboxylic acids, succinic acid, carbonic acid, glutaric acid, decanoic acid, and ascorbic acid. In some embodiments, the composition comprises an inorganic acid selected from, for example, hydrochloric acid, sulfuric acid, nitric acid, phosphoric acid, boric acid, perchloric acid, hydrofluoric acid, hydrobromic acid, and sulfonic acid. Preferably, the acid is present in an amount suitable for adjusting the pH of the composition or the environment to which it is applied to 6.8 or lower, preferably, 5.0 or lower, more preferably, 4.8 or lower.

In certain embodiments, the composition comprises a chelating agent including, but are not limited to, ethylenediaminetetraacetic acid (EDTA), nitrilotriacetic acid (NTA), a phosphonate, succimer (DMSA), diethylenetriaminepentaacetate (DTPA), N-acetylcysteine, n-hydroxyethylethylenediaminetriacetic acid (HEDTA), organic acids with more than one coordination group (e.g., rubeanic acid), STPP (sodiumtripolyphosphate, Na5P3010), trisodium phosphate (TSP), water, carbohydrates, organic acids with more than one coordination group (e.g., citric acid), lipids, steroids, amino acids or related compounds (e.g., glutathione), peptides, phosphates, nucleotides, tetrapyrrols, ferrioxamines, ionophores, orphenolics, sodium citrate, sodium gluconate, ethylenediamine disuccinic acid (EDDS), iminodisuccinic acid (IDS), L-glutamic acid diacetic Acid (GLDA), GLDA-Na4, methyl glycindiacetic acid (MGDA), polyaspartic acid (PASA), hemoglobin, chlorophyll, lipophilic β-diketone, and (14,16)-hentriacontanedione, ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), trimethyl glycine (TMG), Tiron, or any combination thereof.

In one embodiment, the composition comprises one or more fatty acids. The fatty acids can be produced by the microorganisms of the composition, and/or produced separately and included as an additional component. In certain preferred embodiments, the fatty acid is a saturated long-chain fatty acid, having a carbon backbone of 14-20 carbons, such as, for example, myristic acid, palmitic acid, or stearic acid. In some embodiments, a combination of two or more saturated long-chain fatty acids is included in the composition. In some embodiments, a saturated long-chain fatty acid can inhibit methanogenesis and/or increase cell membrane permeability of methanogens.

In certain embodiments, the composition comprises one or more enzymes that help digest food sources into smaller units, such as volatile fatty acids (e.g., propionate, acetate, butyrate), glucose and amino acids. These enzymes can be produced by the microorganisms of the composition, and/or produced separately and included as an additional component. Non-limiting examples of digestive enzymes include amylases, maltases, lactases, lipases, proteases, sucrases and cellulases.

In some embodiments, the composition can comprise additional components known to reduce methane, such as, for example, nitrates (e.g., calcium nitrate, ammonium nitrate, sodium nitrate, potassium nitrate, and magnesium nitrate); seaweed (e.g., Asparagopsis taxiformis and/or Asparagopsis armata); kelp; nitrooxypropanols (e.g., 3-nitrooxypropanol and/or ethyl-3-nitrooxypropanol); anthraquinones; ionophores (e.g., monensin and/or lasalocid); polyphenols (e.g., saponins, tannins); Yucca schidigera extract (steroidal saponin-producing plant species); Quillaja saponaria extract (triterpenoid saponin-producing plant species); organosulfurs (e.g., garlic extract); flavonoids (e.g., quercetin, rutin, kaempferol, naringin, and anthocyanidins; bioflavonoids from green citrus fruits, rose hips and black currants); carboxylic acid; and/or terpenes (e.g., d-limonene, pinene and citrus extracts).

In one embodiment, the composition can comprise one or more biosurfactants. Biosurfactants are a structurally diverse group of surface-active substances produced by microorganisms, which are biodegradable and can be efficiently produced using selected organisms on renewable substrates. All biosurfactants are amphiphiles. They consist of two parts: a polar (hydrophilic) moiety and non-polar (hydrophobic) group. The common lipophilic moiety of a biosurfactant molecule is the hydrocarbon chain of a fatty acid, whereas the hydrophilic part is formed by ester or alcohol groups of neutral lipids, by a carboxylate group of fatty acids or amino acids (or peptides), an organic acid in the case of flavolipids, or, in the case of glycolipids, by a carbohydrate.

Due to their amphiphilic structure, biosurfactants increase the surface area of hydrophobic water-insoluble substances, increase the water bioavailability of such substances, and change the properties of bacterial cell surfaces. Biosurfactants accumulate at interfaces, thus reducing interfacial tension and leading to the formation of aggregated micellar structures in solution. Safe, effective microbial biosurfactants reduce the surface and interfacial tensions between the molecules of liquids, solids, and gases. The ability of biosurfactants to form pores and destabilize biological membranes permits their use as antibacterial, antifungal, and hemolytic agents.

Advantageously, in certain embodiments, biosurfactants can help disrupt and/or penetrate biofilms for increased effectiveness of antibacterial compounds.

Biosurfactants according to the subject invention can include, for example, glycolipids, lipopeptides, flavolipids, phospholipids, fatty acid esters, and high molecular weight polymers such as lipoproteins, lipopolysaccharide-protein complexes, and polysaccharide-protein-fatty acid complexes.

In one embodiment, the biosurfactant is a glycolipid. Glycolipids can include, for example, sophorolipids, rhamnolipids, cellobiose lipids, mannosylerythritol lipids and trehalose lipids. In one embodiment, the biosurfactant is a lipopeptide. Lipopeptides can include, for example, surfactin, iturin, arthrofactin, viscosin, fengycin, and lichenysin. In certain embodiments, a mixture of biosurfactants is used.

In one embodiment, the biosurfactant has been purified from the fermentation medium in which it was produced. Alternatively, in one embodiment, the biosurfactant is utilized in crude form comprising fermentation broth resulting from cultivation of a biosurfactant-producing microbe. This crude form biosurfactant solution can comprise from about 0.001% to 99%, from about 25% to about 75%, from about 30% to about 70%, from about 35% to about 65%, from about 40% to about 60%, from about 45% to about 55%, or about 50% pure biosurfactant, along with residual cells and/or nutrients.

In one embodiment, the composition comprises a saponin at 1 to 10 ml/L, or 2 to 6 ml/L of ruminal fluid. Saponins are natural surfactants that are found in many plants and that exhibit similar characteristics to microbial biosurfactants, for example, self-association and interaction with biological membranes. There are three basic categories of saponins, including triterpenoid saponins, steroidal saponins, and steroidal glycoalkaloids.

Some well-known triterpenoid saponin-accumulating plant families include the Leguminosae, Amaranthaceae, Apiaceae, Caryophyllaceae, Aquifoliaceae, Araliaceae, Cucurbitaceae, Berberidaceae, Chenopodiaceae, Myrsinaceae and Zygophyllaceae, among many others. Quillaja and legumes such as soybeans, beans and peas are a rich source of triterpenoid saponins. The steroidal saponins are typically found in members of the Agavaceae, Alliaceae, Asparagaceae, Dioscoreaceae, Liliaceae, Amaryllidaceae, Bromeliaceae, Palmae and Scrophulariaceae families and accumulate in abundance in crop plants such as yam, alliums, asparagus, fenugreek, yucca, and ginseng. The steroidal glycoalkaloids are commonly found in members of the Solanaceae family including tomato, potato, aubergines and capsicum.

In one embodiment, the composition can comprise one or more biocidal compounds. The biocidal substances can be, for example, antibiotics, including, for example, penicillins (such as penicillin G, penicillin V, ampicillin, amoxicillin, bacampicillin, carbenicillin, carbenicillin indanyl, ticarcillin, azlocillin, mezlocillin, methicillin, piperacillin, and the like), tetracyclines (such as chlortetracycline, oxytetracycline, methacycline, doxycycline, minocycline and the like), cephalosporins (such as cefadroxil, cephalexin, cephradine, cephalothin, cephapirin, cefazolin, cefaclor, cefamandole, cefonicid, cefoxitin, cefotetan, cefuroxime, cefuroxime axetil, cefinetazole, cefprozil, loracarbef, ceforanide, cefepime, cefoperazone, cefotaxime, ceftizoxime, ceftriaxone, ceftazidime, cefixime, cefpodoxime, ceftibuten, and the like), fluoroquinolones (e.g., levofloxacin), quinolones (such as nalidixic acid, cinoxacin, ciprofloxacin and norfloxacin and the like), lincomycins (e.g., clindamycin), macrolides (e.g., erythromycin, azithromycin), sulfones (e.g., dapsone), sulfonamides (e.g., sulfanilamide, sulfadiazine, sulfamethoxazole, sulfisoxazole, sulfacetamide, bactrim), lipopeptides (e.g., daptomycin), polypeptides (e.g., bacitracin), glycopeptides (e.g., vancomycin), aminoglycosides (e.g., streptomycin, gentamicin, tobramycin, amikacin, netilmicin, kanamycin, and the like), nitoimidazoles (e.g., metronidazole) and/or carbapenems (e.g., thienamycin).

In some embodiments, the biocidal substances can include essential oils, botanicals, or other plant extracts with bactericidal and/or anti-bacterial effects. These can include oils/extracts at a concentration between 1-10% volume/volume (extract/invention), horseheal (Inula helenium, L. Asteraceae, elecampane), rose (Rosa damascena L., Rosaceae), lavender (Lavandula angustifolia L., Labiatae), chamomile (Matricaria recutica L., Asteraceae), orange (Rutaceae), grapefruit (Citrus paradisi), eucalyptus (Eucalyptus globulus L.,Myrtaceae), geranium (Geranium robertianum L., Geraniaceae), juniper (Juniperus communis L., Cupressaceae), citrus (Citrus sinensis L., Rutaceae), tea tree (Melaceuca alternifolia), manuka bush (Leptospermum scoparium), neem tree (Azadirachta indica, A. Juss), tea plant (Camellia sinensis), rosemary (Rosmarinus officinalis L., Lamiaceae), lemon, oregano, cinnamon, eucalyptus, citronella, and thyme oils.

Other known biocides, including non-therapeutic biocides, can also be utilized, such as alcohols, aldehydes, chlorine, and chlorine-releasing agents (e.g., sodium hypochlorite, chlorhexidine, chlorhexidine gluconate), iodine, peroxygen compounds (e.g., hydrogen peroxide, peracetic acid), phenolic type compounds, quaternary ammonium compounds (e.g., benzalkonium chloride), bases (e.g., sodium hydroxide, potassium hydroxide, sodium carbonate), and acids (e.g., mineral and organic acids).

In one embodiment, the subject composition can comprise additional nutrients to supplement an animal's diet and/or promote health and/or well-being in the animal, such as, for example, sources of amino acids (including essential amino acids), peptides, proteins, vitamins, microelements, fats, fatty acids, lipids, carbohydrates, sterols, enzymes, prebiotics, and minerals. In some embodiments, the microorganisms of the composition produce and/or provide these substances.

In one embodiment, the subject composition can comprise one or more additional substances and/or nutrients to supplement the needs of the beneficial microorganism of the composition and/or to supplement the needs of the human, animal, or plant to which the composition is administered. These can include, for example, sources of amino acids (including essential amino acids), peptides, proteins, vitamins, microelements, fats, fatty acids, lipids, carbohydrates, sterols, enzymes, and minerals such as calcium, magnesium, phosphorus, potassium, sodium, chlorine, sulfur, chromium, cobalt, copper, iodine, iron, manganese, molybdenum, nickel, selenium, and zinc. In some embodiments, the microorganisms of the composition produce and/or provide these substances.

Preferred compositions comprise vitamins and/or minerals in any combination. Vitamins for use in a composition of this invention can include for example, vitamins A, E, K3, D3, B1, B3, B6, B12, biotin, folic acid, panthothenic acid, nicotinic acid, choline chloride, inositol, and para-amino-benzoic acid. Minerals can include, for example, such as calcium, magnesium, phosphorus, potassium, sodium, chlorine, sulfur, chromium, cobalt, copper, iodine, iron, manganese, molybdenum, nickel, selenium, and zinc. Other components may include, but are not limited to, antioxidants, beta-glucans, bile salt, cholesterol, enzymes, carotenoids, and many others. Typical vitamins and minerals are those, for example, recommended for daily consumption and in the recommended daily amount (RDA), although precise amounts can vary.

In certain embodiments, the composition can further comprise one or more carriers and/or excipients suitable for internal or external delivery of the composition to a human or animal subject, a plant or to an inert surface.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (such as, e.g., Tween 80, Polysorbate 80), colloids, dispersion media, vehicles, fillers (e.g., MCC), chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, coatings, preservatives (such as, e.g., Thimerosal, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of carriers and/or excipients in the field of drugs and supplements, agriculture, and cleaning compositions is well known. Except for any conventional media or agent that is incompatible with the components of the subject compositions, its use in the subject compositions may be contemplated.

In certain embodiments, the composition comprises a filler, such as microcrystalline cellulose (MCC).

In certain embodiments, the composition can comprise glucose (e.g., in the form of molasses), glycerol and/or glycerin, as, or in addition to, an osmoticum substance, to promote osmotic pressure during storage and transport of a dry product.

The compositions can be formulated into preparations in, for example, solid, semi-solid, liquid or gaseous forms, such as tablets, capsules, pressed pellets, powders, granules, ointments, gels, lotions, solutions, suppositories, drops, patches, injections, inhalants, aerosols, orally-consumable food and beverage products, suspensions, concentrates, and other preparations as suitable for a particular application.

In one embodiment, the composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the composition can be made into aerosol formulations so that, for example, it can be nebulized or inhaled. Suitable formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the composition can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

In one embodiment, the composition can be formulated for direct administration into the digestive system of a human or animal subject via, for example, injection and/or endoscopy, for example, as a solution or suspension. The solution or suspension can comprise suitable non-toxic, enterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Water or saline solutions and aqueous dextrose and glycerol solutions may be preferably employed as carriers, particularly for enterally-injectable solutions.

In one exemplary embodiment, the microbe-based composition can be formulated for oral administration via an orally-consumable product. Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable drug can be formulated into an orally consumable product, and an orally consumable product can comprise an orally deliverable drug, the two terms are not meant to be used interchangeably herein.

Orally consumable products include all substances or products intended to be ingested by humans or animals in a processed, semi-processed, or unprocessed state. This also includes substances that are added to orally consumable products (particularly food and drug products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

In certain embodiments, the composition is formulated for application to soil, seeds, whole plants, or plant parts (including, but not limited to, roots, tubers, stems, stalks, buds, flowers and leaves). In certain embodiments, the composition is formulated as, for example, liquid, dust, granules, microgranules, pellets, wettable powder, flowable powder, emulsions, microcapsules, oils, or aerosols.

To improve or stabilize the effects of the composition, it can be blended with suitable adjuvants and then used as such or after dilution, if necessary. In preferred embodiments, the composition is formulated as a liquid, a concentrated liquid, or as dry powder or granules that can be mixed with water and other components to form a liquid product.

Further components can be added to the composition, for example, buffering agents, carriers, viscosity modifiers, preservatives, tracking agents, biocides, other microbes, other microbe-based compositions, surfactants, emulsifying agents, lubricants, solubility controlling agents, pH adjusting agents, preservatives, stabilizers, ultra-violet light resistant agents, essential oils, botanical extracts, cross-linking agents, chelators, fatty acids, alcohols, reducing agents, syndetics, enzymes, dyes, colorants, fragrances, antimicrobial compounds, antibiotics, foaming agents, foam reducers, polymers, thickeners, and chelators.

### EXEMPLARY EMBODIMENTS

In some embodiments, the composition of the subject invention comprises:
A) one or more microorganisms (yeasts, fungi and/or bacteria) capable of capturing iron and/or producing an iron-capturing growth by-product, wherein preferably at least one of the one or more microorganisms is a Bacillus sp., and wherein even more preferably, the Bacillus sp. is B. subtilis NRRL B-68031 or B. amyloliquefaciens NRRL B-67928;
B) an iron-capturing substance selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, siderite, transferrin, enterobactin, bacillibactin, vibriobactin, azotobactin, aminochelin, pyoverdine, yersiniabactin, agrobactin, staphyloferrin, hydroxamate, catecholate, salicylate, carboxylate, mugineic acid, ferulic acid, caffeic acid, enterobactin, pyoverdine, protochelin, pyochelin, vibrioferrin and/or nicotianamine;
C) a carrier/excipient including solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents, solubilizers (such as, e.g., Tween 80, Polysorbate 80), colloids, dispersion media, vehicles and/or fillers (e.g., MCC);
D) one or more acids selected from, for example, acetic acid, citric acid, formic acid, lactic acid, malic acid, oxalic acid, tartaric acid, uric acid, propionic acid, butyric acid, sorbic acid, fumaric acid, benzoic acid, hydrofluoric acid, caproic acid, salicylic acid, gluconic acid, pyruvic acid, adipic acid, trichloroacetic acid, glycolic acid, cinnamic acid, carboxylic acids, succinic acid, carbonic acid, glutaric acid, decanoic acid, ascorbic acid, hydrochloric acid, sulfuric acid, nitric acid, phosphoric acid, boric acid, perchloric acid, hydrofluoric acid, hydrobromic acid, and sulfonic acid, in an amount suitable for adjusting the pH of the composition or the environment to which it is applied to 6.8 or lower, preferably, 5.0 or lower, more preferably, 4.8 or lower;
E) a biosurfactant selected from sophorolipids, rhamnolipids, cellobiose lipids, mannosylerythritol lipids, trehalose lipids, surfactin, iturin, arthrofactin, viscosin, fengycin, and lichenysin; and/or
F) a biocidal compound, such as an antibiotic, a biosurfactant or a botanical extract.

In some embodiments, the composition comprises each of components A-F. In some embodiments, the composition comprises any combination of A-F, or any one of A-F individually.

In some embodiments, the composition comprises spore, vegetative and/or biofilm-form B. subtilis NRRL B-68031 or B. amyloliquefaciens NRRL B-67928; and pulcherrimin and/or pulcherriminic acid.

In some embodiments, the composition comprises spore, vegetative and/or biofilm-form B. subtilis NRRL B-68031 or B. amyloliquefaciens NRRL B-67928; and a carrier/excipient.

In some embodiments, the composition comprises spore, vegetative and/or biofilm-form B. subtilis NRRL B-68031 or B. amyloliquefaciens NRRL B-67928; and one or more acids listed in point D).

In some embodiments, the composition comprises spore, vegetative and/or biofilm-form B. subtilis NRRL B-68031 or B. amyloliquefaciens NRRL B-67928; pulcherrimin and/or pulcherriminic acid; and a carrier/excipient.

In some embodiments, the composition comprises spore, vegetative and/or biofilm-form B. subtilis NRRL B-68031 or B. amyloliquefaciens NRRL B-67928; pulcherrimin and/or pulcherriminic acid; a carrier; and one or more acids listed in point D).

In some embodiments, the composition comprises spore, vegetative and/or biofilm-form B. subtilis NRRL B-68031 or B. amyloliquefaciens NRRL B-67928; pulcherrimin and/or pulcherriminic acid; a carrier; and a biosurfactant.

In some embodiments, the composition comprises spore, vegetative and/or biofilm-form B. subtilis NRRL B-68031 or B. amyloliquefaciens NRRL B-67928; pulcherrimin and/or pulcherriminic acid; a carrier; and a biocidal compound.

Production of Microorganisms and/or Microbial Growth By-Products

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites, residual nutrients and/or intracellular components.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In one embodiment, one or more biostimulants may also be included, meaning substances that enhance the rate of growth of a microorganism. Biostimulants may be species-specific or may enhance the rate of growth of a variety of species.

In some embodiments, the method for cultivation may further comprise adding an antimicrobial in the medium before, and/or during the cultivation process.

In certain embodiments, an antibiotic can be added to a culture at low concentrations to produce microbes that are resistant to the antibiotic. The microbes that survive exposure to the antibiotic are selected and iteratively re-cultivated in the presence of progressively higher concentrations of the antibiotic to obtain a culture that is resistant to the antibiotic. This can be performed in a laboratory setting or industrial scale using methods known in the microbiological arts. In certain embodiments, the amount of antibiotic in the culture begins at, for example, 0.0001 ppm and increases by about 0.001 to 0.1 ppm each iteration until the concentration in the culture is equal to, or about equal to, the dosage that would typically be applied to a iron-capturing pathogen.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The biomass content of the fermentation medium may be, for example, from 5 g/l to 180 g/l or more, or from 10 g/l to 150 g/l. The cell concentration may be, for example, at least 1×10⁹, 1×10¹⁰, 1×10¹¹, 1×10¹² or 1×10¹³ cells per gram of final product.

The microbial growth by-product produced by microorganisms of interest may be retained in the microorganisms or secreted into the growth medium. The medium may contain compounds that stabilize the activity of microbial growth by-product.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

### Preparation of Microbe-Based Products

A “microbe-based product,” is a product to be applied in practice to achieve a desired result. The microbe-based product can be simply a microbe-based composition harvested from a microbe cultivation process. Alternatively, a microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

One microbe-based product of the subject invention is simply the fermentation medium containing a microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

The microorganisms in the microbe-based product may be in an active or inactive form. Furthermore, the microorganisms may be removed from the composition, and the residual culture utilized. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium (e.g., broth or solid substrate) resulting from the microbial growth can be removed from the growth vessel and transferred via, for example, piping for immediate use.

In one embodiment, the microbe-based product is simply the growth by-products of the microorganism. For example, biosurfactants produced by a microorganism can be collected from a submerged fermentation vessel in crude form, comprising, for example about 50% pure biosurfactant in liquid broth.

In other embodiments, the microbe-based product (microbes, medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting, for example, the yeast fermentation product, from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Examples of such additives include surfactants, emulsifying agents, lubricants, buffering agents, solubility controlling agents, pH adjusting agents, preservatives, stabilizers and ultra-violet light resistant agents.

In one embodiment, the product may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, or sodium biphosphate, can be included in the formulation.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Methods for Inhibiting an Iron-Capturing Pathogen

In preferred embodiments, the subject invention provides methods for controlling iron-capturing pathogens, wherein a composition comprising an iron-capturing ingredient is introduced into an environment in which the pathological microorganism is growing. Thus, the subject invention provides methods for controlling a pathogen, wherein the composition applied to the pathogen's environment outcompetes the pathogen for iron resources. The methods can be utilized in environments including, for example, human and/or animal subjects, soil, plants, water and/or inanimate surfaces.

Advantageously, in certain embodiments, the methods result in capture of iron present in the pathogen's environment, thereby inhibiting its pathogenicity via iron limitation. Thus, in preferred embodiments, the methods result in a direct inhibition of pathogens, increased out-competition of pathogens by beneficial microorganisms, disruption of pathogenic biofilms, and/or disruption of one or more biological pathways in which the pathogen utilizes iron for growth, reproduction and/or virulence.

Non-limiting examples of bacterial pathogens that can utilize one or more iron acquisition systems to enhance infectious virulence include Escherichia spp. (e.g., E. coli), Vibrio spp. (e.g., V. cholerae, V. vulnificus), Shigella spp. (e.g., S. flexneri, S. dysenteriae), Salmonella spp. (e.g., S. enterica), Streptococcus spp. (e.g., S. puyogenes, S. agalactiaei, Group A Streptococcus), Yersinia spp. (e.g., Y. pestis), Haemophilus spp. (e.g., H. influenzae), Klebsiella spp. (e.g. K. pneumoniae), Staphylococcus spp. (e.g., S. aureus), Mycobacterium spp. (e.g., M. tuberculosis), Neisseria spp. (e.g., N. meningitidis, N. gonorrhoeae), Bartonella spp. (e.g., B. quintana), Bacillus spp. (e.g., B. anthracis, B. cereus), Serratia spp. (e.g., S. marcescens), Pseudomonas spp. (e.g., P. aeruginosa), Legionella spp. (e.g., L. pneumophila), Meningococcus spp., Brucella spp. (e.g., B. abortus), Listeria spp. (e.g., L. monocytogenes), Acinetobacter spp. (e.g., A. baumannii), Francisella spp. (e.g., F. tularensis), and/or Haemophilus spp. (e.g., H. influenzae).

In some embodiments, the method comprises applying an iron-capturing ingredient according to the subject invention alongside an acid, wherein the acid modulates the pH of the composition or the environment to 6.8 or lower, preferably 5.0 or lower, more preferably 4.8 or lower. The acid can be an organic acid selected from, for example, acetic acid, citric acid, formic acid, lactic acid, malic acid, oxalic acid, tartaric acid, uric acid, propionic acid, butyric acid, sorbic acid, fumaric acid, benzoic acid, hydrofluoric acid, caproic acid, salicylic acid, gluconic acid, pyruvic acid, adipic acid, trichloroacetic acid, glycolic acid, cinnamic acid, carboxylic acids, succinic acid, carbonic acid, glutaric acid, decanoic acid, and ascorbic acid. The acid can also be an inorganic acid selected from, for example, hydrochloric acid, sulfuric acid, nitric acid, phosphoric acid, boric acid, perchloric acid, hydrofluoric acid, hydrobromic acid, and sulfonic acid.

In certain specific embodiments, the methods comprise administering a composition according to the subject invention to a human or animal subject in need thereof (i.e., a subject infected with an iron-capturing pathogen or at risk of such infection).

Administration to a human or animal subject can be acute or chronic (e.g., hourly, daily, weekly, monthly, etc.) or in combination with other agents. The subject compositions can be administered by any route of administration provided they are formulated for such a route. In this way, the therapeutic effects attainable by the methods and compositions of the invention can be, for example, systemic, local, tissue-specific, etc. depending on the specific needs of a given application of the invention.

In one embodiment, the compositions are administered orally, via injection (which includes intravenously, intraperitoneally, intramuscularly, intrathecally, or subcutaneously), via the skin (e.g., through a patch or directly onto the skin for local or systemic effects), sublingually, buccally, rectally, or vaginally. Furthermore, the compositions can be sprayed into the nose for absorption through the nasal membrane, nebulized, inhaled via the mouth or nose, or administered in the eye or ear.

In some embodiments, the methods can further comprise applying materials to enhance the growth of the microorganisms of the subject composition at the time of application (e.g., adding nutrients and/prebiotics). In one embodiment, the nutrient sources can include, for example, sources of magnesium, phosphate, nitrogen, potassium, selenium, calcium, sulfur, iron, copper, zinc, proteins, vitamins and/or carbon. In certain embodiments, the iron-capturing pathogen can be fed a source of prebiotics, which can include, for example, dry animal fodder, straw, hay, alfalfa, grains, forage, grass, fruits, vegetables, oats, and/or crop residue.

In certain embodiments, the methods comprise adding the composition to drinking water and/or animal feed as a dietary supplement. The dietary supplement can have any suitable form such as a gravy, drinking water, beverage, yogurt, powder, granule, paste, suspension, chew, morsel, liquid solution, treat, snack, pellet, pill, capsule, tablet, sachet, or any other suitable delivery form. The dietary supplement can comprise the subject microbe-based compositions, as well as optional compounds such as vitamins, minerals, probiotics, prebiotics, and antioxidants. In some embodiments, the dietary supplement may be admixed with a feed composition or with water or other diluent prior to administration to the animal.

In some embodiments, the composition is applied to a grazing field or pasture as well as to the drinking water and/or feed.

In certain specific embodiments, the methods comprise administering a composition according to the subject invention to a plant or soil environment in need thereof (i.e., a plant or soil environment infected with an iron-capturing pathogen or at risk of such infection). In some embodiments, multiple plants and/or their surrounding environments are treated according to the subject methods.

Application can include contacting the composition directly with a plant, plant part, and/or the plant's surrounding environment (e.g., the soil or the rhizosphere). The microbe-product can be applied as a seed treatment, or to the soil surface, or to the surface of a plant or plant part (e.g., to the surface of the roots, tubers, stems, flowers, leaves, fruit, or flowers). It can be sprayed, poured, sprinkled, injected or spread as liquid, dry powder, dust, granules, microgranules, pellets, wettable powder, flowable powder, emulsions, microcapsules, oils, gels, pastes or aerosols.

As used herein, a plant's “surrounding environment” means the soil and/or other medium in which the plant is growing, which can include the rhizosphere. In certain embodiments, the surrounding environment does not extend past, for example, a radius of at least 5 miles, 1 mile, 1,000 feet, 500 feet, 300 feet, 100 feet, 10 feet, 8 feet, or 6 feet from the plant.

In a specific embodiment, the composition is contacted with one or more roots of the plant. The composition can be applied directly to the roots, e.g., by spraying or dunking the roots, and/or indirectly, e.g., by administering the composition to the soil in which the plant grows (e.g., the rhizosphere). The composition can be applied to the seeds of the plant prior to or at the time of planting, or to any other part of the plant and/or its surrounding environment.

In certain embodiments, the composition is applied to the soil surface without mechanical incorporation. The beneficial effect of the soil application can be activated by rainfall, sprinkler, flood, or drip irrigation, and subsequently delivered to, for example, the roots of plants.

Plants and/or their environments can be treated at any point during the process of cultivating the plant. For example, the immune supplement composition can be applied to the soil prior to, concurrently with, or after the time when seeds are planted therein. It can also be applied at any point thereafter during the development and growth of the plant, including when the plant is flowering, fruiting, and during and/or after abscission of leaves.

Furthermore, the composition can be applied prior to infection of a plant by a pest or pathogen, or after infection has occurred.

In one embodiment, the method can be used in a large scale agricultural setting. The method can comprise administering the composition into a tank connected to an irrigation system used for supplying water, fertilizers or other liquid compositions to a crop, orchard or field. Thus, the plant and/or soil surrounding the plant can be treated with the composition via, for example, soil injection, soil drenching, or using a center pivot irrigation system, or with a spray over the seed furrow, or with sprinklers or drip irrigators. Advantageously, the method is suitable for treating hundreds of acres of crops, orchards or fields at one time.

In one embodiment, the method can be used in a smaller scale setting, such as in a home garden or greenhouse. In such cases, the method can comprise spraying a plant and/or its surrounding environment with the composition using a handheld lawn and garden sprayer. The composition can be mixed with water, and optionally, other lawn and garden treatments, such as fertilizers and pesticides. The composition can also be mixed in a standard handheld watering can and poured onto soil.

The methods can comprise adding materials to enhance microbe growth during application (e.g., adding nutrients and/or prebiotics to promote microbial growth). In one embodiment, the nutrient sources can include, for example, sources of nitrogen, potassium, phosphorus, magnesium, proteins, vitamins and/or carbon. In one embodiments, the prebiotics can include, for example, kelp extract, fulvic acid, humate and/or humic acid. To improve or stabilize the effects of the composition, it can be blended with suitable adjuvants and then used as such or after dilution if necessary.

In certain specific embodiments, the methods comprise administering a composition according to the subject invention to water and/or to an inert or inanimate surface having an iron-capturing pathogen therein or thereon.

The composition can simply be mixed into a source of water, for example, a pool, water treatment facility, animal drinking container, lake, pond, or fish farm. In the case of biofilms in water sources, such as man-made pools, water treatment facilities and other water enclosures, the composition can be applied to the surfaces within the pool or enclosure before the water is present.

The composition can be applied to a surface by spraying using, for example, a spray bottle or a pressurized spraying device. The composition can also be applied to a surface using a cloth or a brush, wherein the composition is rubbed, spread or brushed onto the surface. Furthermore, the composition can be applied to the surface by dipping, dunking or submerging the surface into a container having the composition therein.

In one embodiment, the surface is allowed to soak with the composition thereon for a sufficient time to remove the pathogen. For example, soaking can occur for up to 5 minutes to 24 hours or more, as needed.

In one embodiment, the method further comprises the step of removing the composition and pathogen from the surface. This can be achieved by, for example, rinsing or spraying water onto the surface, and/or rubbing or wiping the surface with a cloth until the composition and pathogen have been freed from the surface. Rinsing or spraying with water can be performed before and/or after rubbing or wiping the surface with a cloth. In some embodiments, the spraying is performed under elevated pressure and/or elevated temperature.

In another embodiment, mechanical methods can be used to remove the pathogen and/or composition from the surface after application of the composition. For example, a sandblaster, agitator, drill, hammer, sandpaper, or scraper can be used for freeing contaminants from surfaces that are particularly difficult to remove due to, for example, the amount of contaminant or the type of contaminant.

The methods of the present invention can be used for treating and/or preventing a disease or infection caused by a pathogenic microorganism, including pathogenic biofilms. Examples of such diseases include, for example, diseases and/or infections caused by Agrobacterium spp. (e.g., A. tumefaciens), Dickeya spp. (e.g., D. dadantii), Erwinia spp. (e.g., E. amylovora, E. carotovora), Escherichia spp. (e.g., E. coli), Vibrio spp. (e.g., V. cholerae, V. vulnificus), Shigella spp. (e.g., S. flexneri, S. dysenteriae), Salmonella spp. (e.g., S. enterica, S. enteritidis, S. newport, S. typhimurium, S. javiana), Streptococcus spp. (e.g., S. puyogenes, S. agalactiaei, Group A Streptococcus), Yersinia spp. (e.g., Y. pestis), Haemophilus spp. (e.g., H. influenzae), Klebsiella spp. (e.g. K. pneumoniae), Staphylococcus spp. (e.g., S. aureus), Mycobacterium spp. (e.g., M. tuberculosis), Neisseria spp. (e.g., N. meningitidis, N. gonorrhoeae), Bartonella spp. (e.g., B. quintana), Bacillus spp. (e.g., B. anthracis, B. cereus), Serratia spp. (e.g., S. marcescens), Pseudomonas spp. (e.g., P. aeruginosa, P. syrinage), Helicobacter spp. (e.g., H. pylori), Legionella spp. (e.g., L. pneumophila), Meningococcus spp., Brucella spp. (e.g., B. abortus), Listeria spp. (e.g., L. monocytogenes), Acinetobacter spp. (e.g., A. baumannii), Francisella spp. (e.g., F. tularensis), Ralstonia spp. (e.g., R. solanacearum) and/or Haemophilus spp. (e.g., H. influenzae).

Specific exemplary human and animal conditions include Legionnaires' disease, meningitis, septicemia, anthrax, plague, gonorrhea, endocarditis, osteomyelitis, cholera, bacteremia, tuberculosis, pneumonia, typhoid fever, parathyphoid fever, food poisoning, urinary tract infections, listeriosis, lyme disease, Strep throat, Staph infections, MRSA, gastric hypochlorhydria and iron deficiency anemia.

Specific exemplary plant conditions include soft rot, wildfire disease, blight, fruit necrotic spots, bacterial speck, crown gall tumors, wilt, stem rot, and fire blight.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—PH Growth Testing of B4 Strain

The B4 strain was spread on Tryptic Soy Agar (TSA) at a neutral (6.8) and acidic (4.8) pH to look for differences in growth, with the goal of determining how it would behave in the various pH environments within a cow's digestive system.

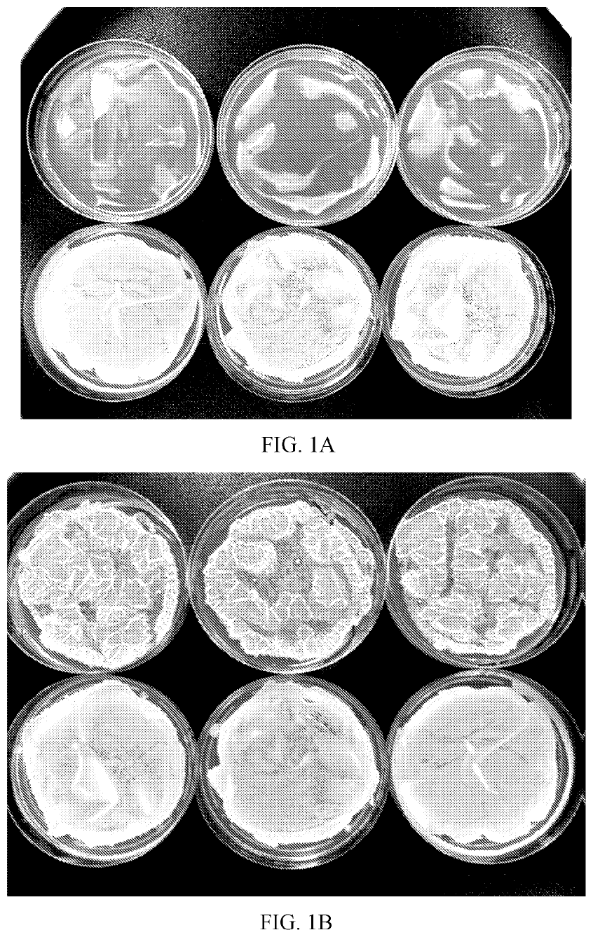

Growth at neutral pH (6.8) was faster within 24 hours compared to the acidic plates, but at 48 hours, the growth on pH 4.8 agar was equal or greater. FIGS. 1A-1B. Additionally, a significant amount of exopolysaccharide (EPS) was produced when grown on an acidic agar. FIG. 1C. This is a result of environmental stress.

The dried B4 spores were also added in sterile PBS adjusted to pH 2.8 and left for 24 hours. The same was then plated on neutral (6.8) TSA plates. A lawn of growth was present, but no EPS was produced (similar to pH 6.8 plates from FIG. 1). This shows that dried spores exposed to an overall harsh environment were still intact and viable. Overall, the pH of the growing media/environment influences EPS production.

### Example 2—EPS

B4 was grown in a liquid medium at pH 4.8 specifically for the production of EPS. The assumed EPS was isolated out of the culture and purified.

FTIR analysis confirmed the purified sample to be an EPS and HPLC analysis confirmed the existence of a large peak denoting a sugar oligomer. A C—O bond was also observed via UV absorption.

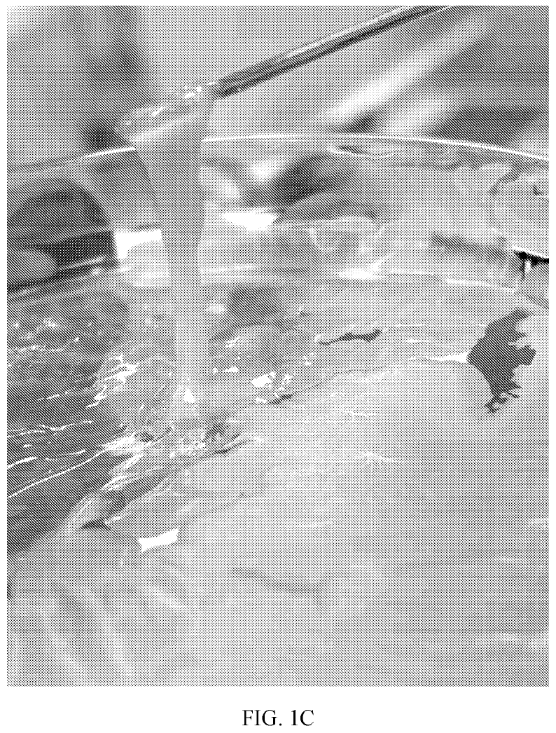

Additionally, and surprisingly, when the culture was processed for EPS extraction, there was a purple stripe present in the cell pellet. When the EPS purification was completed, the sample was a pink color, suggesting the presence of pulcherrimin or pulcherriminic acid. FIG. 2.

### Example 3—Enzyme Assay Testing-Amylase

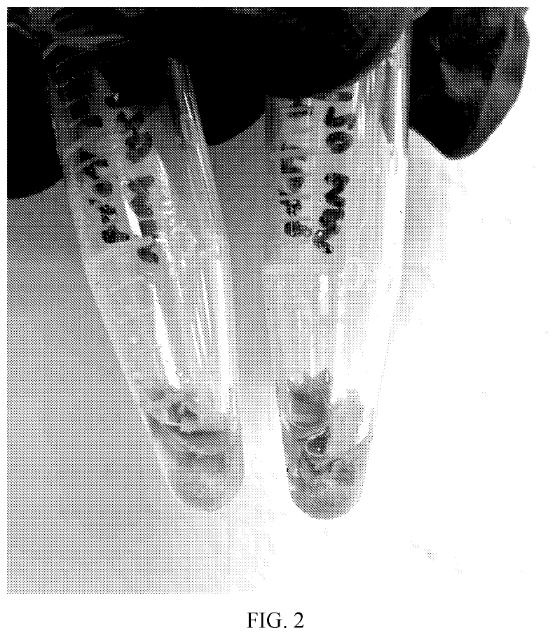

Amylase is an enzyme that hydrolyzes the glycosidic bonds in starch molecules by converting complex carbohydrates to simple sugars. Agar, a starch, was inoculated with B4 and incubated for growth. FIG. 3. After running the plate assay, an orange color around the bacterial growth was observed, indicating the breakdown of starch. This is a positive amylase test.

### Example 4—Enzyme Assay Testing-Cellulase

Cellulases are enzymes that convert cellulose to glucose. B4 was tested for cellulase activity using carboxymethylcellulose agar (CMCA) media plates.

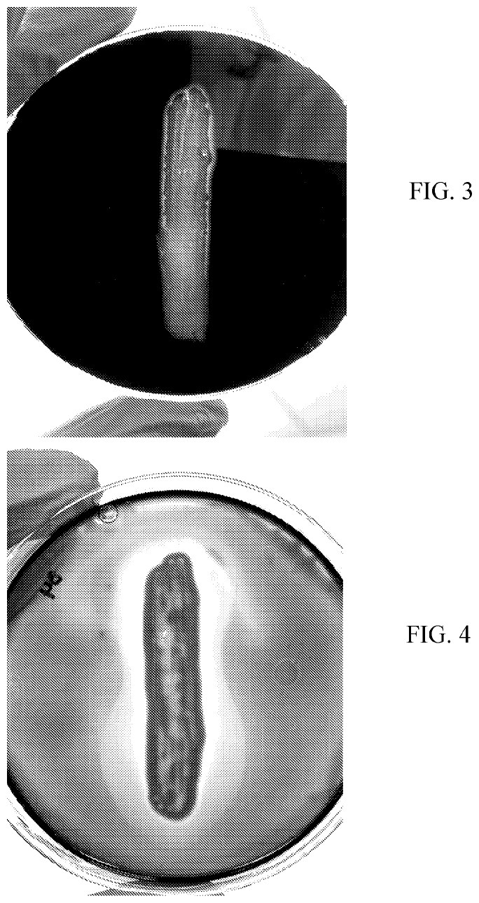

B4 was grown in tryptic soy broth with and without cellobiose added. After 48 hours of growth, the liquid culture was streaked onto CMCA plates. After inoculation, and once growth was present on the agar, an iodine solution was introduced to the plates. A yellow zone of clearing around the bacterial growth indicates the breakdown of cellulose and the presence of cellulase enzymes. FIG. 4.

### Example 5—Detection of Siderophores

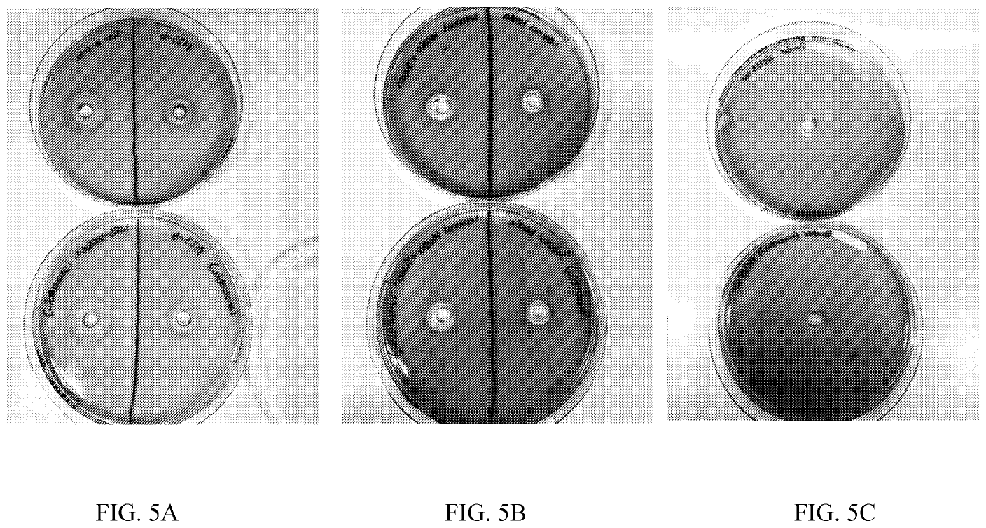

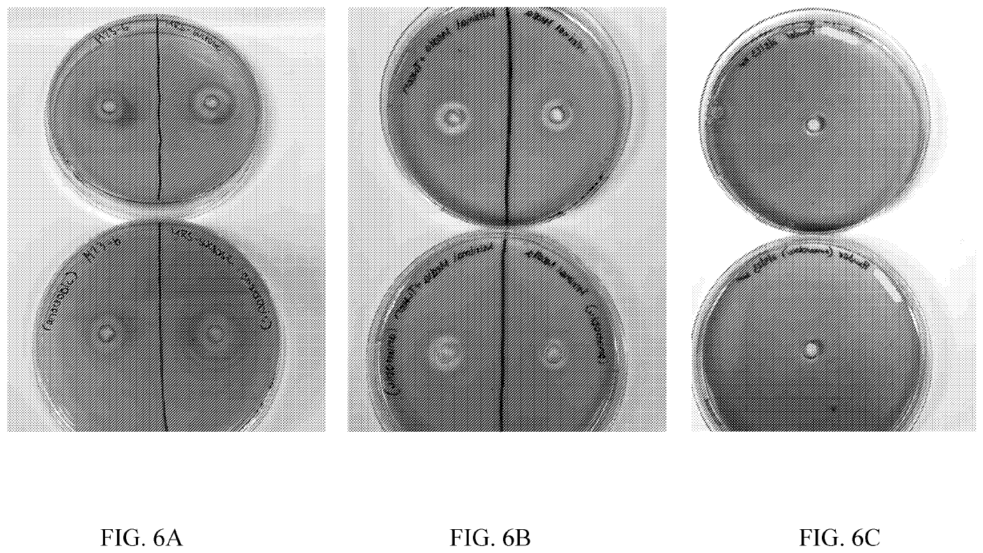

Chrome azurol S (CAS) assay was used for the detection of siderophores from B4 cultures and dried B4 spores. The tests were run in aerobic and anaerobic environments on different growth media: MRS-sucrose, M23-6, minimal media with Tween, and minimal media without Tween. The plates were observed at 6 hours (FIGS. 5A-5C) and 24 hours (FIGS. 6A-6C).

Siderophores scavenge iron from an Fe-CAS-hexadecyltrimethylammonium bromide complex, and the release of the CAS dye results in a color change from blue to orange. Any observable color change on CAS agar plates indicates a qualitative detection of siderophores.

All B4 media cultures tested were positive for siderophore production and activity. MRS-sucrose (the richest media) produced the strongest activity. There were no differences in siderophore activity between aerobic and anaerobic conditions with the four media cultures.

Dried B4 spores produced less siderophore activity compared to the B4 cultures, which may due to spore dormancy. The dried spores performed better in aerobic conditions.

### Example 6—Iron Activity Assay

To understand how B4 interacts with iron, and to determine how iron activity differs between culture media, an iron assay kit (Sigma-Aldrich) was used to determine the concentration of ferrous (Fe²⁺), ferric (Fe³⁺) and total iron present in different B4 cultures.

Iron is released from the sample by the addition of an acidic buffer. Released iron is reacted with chromagen, resulting in a colorimetric (593 nm) product that is proportional to the iron present.

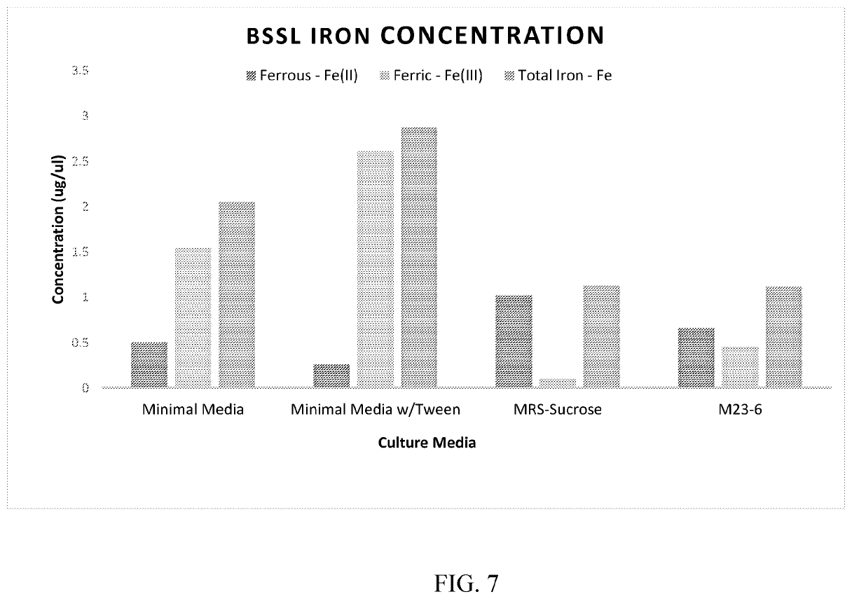

The results in FIG. 7 correlate with the siderophore results reported in FIGS. 5-6, meaning higher siderophore activity correlates with lower total iron levels. For example, MRS-sucrose media is the richest media with the highest siderophore activity and the lowest total iron levels. Additionally, the minimal media used is designed for increased production of pulcherrimin production, which is a ferric chelate. Greater levels of pulcherrimin in the culture should increase ferric and total iron concentrations.

1. A method for controlling an iron-capturing pathogen, wherein the method comprises applying, to the environment of the pathogen, an iron-capturing agent; wherein the iron-capturing agent is am iron-capturing non-pathogenic Bacillus sp. bacterium and/or an iron-capturing growth by-product thereof.

2. The method of claim 1, further comprising applying an acid to the environment to achieve a pH of 6.8 or less.

3. (canceled)

4. The method of claim 1, wherein the pathogenic microorganism is selected from: Agrobacterium spp., Dickeya spp., Erwinia spp., Escherichia spp., Vibrio spp., Shigella spp., Salmonella spp., Streptococcus spp., Yersinia spp., Haemophilus spp., Klebsiella spp., Staphylococcus spp., Mycobacterium spp., Neisseria spp., Bartonella spp., Bacillus spp., Helicobacter spp., Serratia spp., Pseudomonas spp., Legionella spp., Meningococcus spp., Brucella spp., Listeria spp., Acinetobacter spp., Francisella spp., Ralstonia spp. and/or Haemophilus spp.

5. The method of claim 1, wherein the environment is a human or animal subject, a plant or soil environment, water, or an inanimate surface.

6. The method of claim 5, wherein the environment is a human or animal subject, and wherein the composition is administered to the subject orally, via injection, via inhalation, or topically.

7. The method of claim 5, wherein the environment is a plant or soil environment, and wherein the composition is administered to the plant or soil via an irrigation system or via a spray applicator.

8. The method of claim 5, wherein the environment is an inanimate surface, and wherein after the composition is administered to the surface, the composition and the pathogen are removed from the surface.

9-10. (canceled)

11. The method of claim 1, wherein the microorganism is Bacillus subtilis B4 NRRL B-68031 or Bacillus amyloliquefaciens NRRL B-67928.

12. The method of claim 1, wherein the microorganism is administered in spore form, but grows in biofilm form upon contact with an acidic environment having pH 6.8 or less.

13. The method of claim 1, comprising applying a further iron-capturing agent selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and nicotianamine.

14. The method of claim 1, wherein the composition further comprises one or more microbial growth by-products selected from biosurfactants, enzymes, organic acids, fatty acids, amino acids, proteins, peptides, alcohols, polyketides, natural antibiotics, aldehydes, amines, sterols, and vitamins.

15. A composition for controlling iron-capturing pathogens, the composition comprising an iron-capturing agent, wherein the iron-capturing agent is a non-pathogenic Bacillus sp. bacterium, and/or an iron capturing growth by-product thereof.

16. The composition of claim 15, having a pH of 6.8 or less.

17-18. (canceled)

19. The composition of claim 15, wherein the microorganism is Bacillus subtilis B4 NRRL B-68031 or Bacillus amyloliquefaciens NRRL B-67928.

20. The composition of claim 15, wherein the microorganism is in spore form or biofilm form.

21. The composition of claim 15, wherein the composition comprises a further iron-capturing ingredient is-selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine.

22. The composition of claim 15, further comprising a germination enhancer, wherein the germination enhancer is L-alanine, L-leucine, or manganese.

23. The composition of claim 15, wherein the composition comprises a microbial growth by-product selected from biosurfactants, enzymes, organic acids, fatty acids, amino acids, proteins, peptides, alcohols, polyketides, natural antibiotics, aldehydes, amines, sterols, and vitamins.

24. The composition of claim 15, further comprising a nutrient or prebiotic.
